(19) United States
(12) Patent Application Publication
(10) Pub. No.: US 2013/0122559 A1
(43) Pub. Date: May 16, 2013

(54) METHOD FOR SYNTHESIZING POLYHYDROXYALKANOATE USING A MICROORGANISM

(71) Applicants: Chi-Wei Lan, Taipei (TW); Cheng-Yung Chang, Hsinchu (TW)

(72) Inventors: Chi-Wei Lan, Taipei (TW); Cheng-Yung Chang, Hsinchu (TW)

(73) Assignee: YUAN ZE UNIVERSITY, Chung-Li (TW)

(21) Appl. No.: 13/296,588

(22) Filed: Nov. 15, 2011

(51) Int. Cl. C12P 7/62

(52) U.S. Cl. 435/135

(57) ABSTRACT

A method for synthesizing polyhydroxyalkanoate using a microorganism includes providing a Ralstonia eutropha strain; providing a fermentation tank and adding crude glycerol into the fermentation tank; adding a nitrogen source into the fermentation tank to adjust a carbon to nitrogen (C/N) ratio to 20:1 to 150:1; controlling temperature of the fermentation tank to a temperature ranging from 20 to 45° C.; adding 5 to 20% by volume of the Ralstonia eutropha strain into the fermentation tank using a mixing speed of from 100 to 500 rpm, an aeration quantity of 0.5-2 vvm and a concentration of dissolved oxygen of 30 to 80%; fermenting contents of the fermentation tank for 48 to 96 hours to allow the Ralstonia eutropha strain to proliferate and to allow polyhydroxyalkanoate to be synthesized from the crude glycerol within cells of the Ralstonia eutropha strain; and disrupting the cells of the Ralstonia eutropha strain and extracting polyhydroxyalkanoate.

### BACKGROUND OF THE INVENTION

1. Field of the Invention

The present invention relates generally to a method for synthesizing polyhydroxyalkanoate using a microorganism, and more particularly to a method for synthesizing polyhydroxyalkanoate from crude glycerol, which is a byproduct of biodiesel, using a microorganism.

2. The Prior Arts

In view of rapid development of biodiesel, B2 biodiesel has been already developed to full industrial type production in Taiwan in the year of 2009, and the amount of crude glycerol produced from the biodiesel is increased gradually. However, the refining cost of the crude glycerol is high, so that the value-added utilization of crude glycerol from biodiesel becomes an important issue. Accordingly, polyhydroxyalkanoate (PHA) is produced from crude glycerol, and a wide range of high-value-added products can be manufactured from polyhydroxyalkanoate.

In Japan, Doi's research group has focused on the improvement of the synthesis of polyhydroxyalkanoate through strain improvement (such as E. coli and Wauteria eutropha) and gene mutation (such as genes of phaP or phaR). In addition, the increase of culture volume of the strain, and the mass-production of the strain in a fermentation tank, and the industrial testing techniques are all very mature.

Furthermore, in China, the research team directed by Prof. Chen GQ in Tsinghua University not only studied on the gene but also on the strain modification. Furthermore, polyhydroxyalkanoate is produced using a large fermentation tank in some companies. The Beijing 2008 Olympic Games used the biodegradable polymers for the environmental protection, and the achievements of the research on the biodegradable plastics are also extensively reported.

In Korea, the strain improvement and the improvement of the metabolic pathway, and high density fermentation for the synthesis of polyhydroxyalkanoate are deeply researched by the teams of Prof S. Y. Lee. In Germany, Prof. A. Steinbuchel and his research teams decoded the gene sequence of the microorganism of Ralstonia eutropha completely and found the homology genes in genes phaC, phaB, and phaA of the chromosomes. In Britain, a series of polyhydroxyalkanoate polymers, such as 3-hydroxybutyrate(3HB), 3-hydroxyvalerate(3HV), and 4-hydroxybutyrate(4HB) are produced using Bacillus cereus SPV by Prof I. Roy. In Switzerland, polyhydroxyalkanoate is synthesized in a number of transgenic plants that is different from the microorganism system by Prof Witholt. In U.S.A., bio-polyesters are produced using gene recombinant Escherichia coli by Prof F. Srienc. In Australia, the synthesis of polyhydroxyalkanoate using Pseudomonas genus by Prof Rehm of New Zealand based on genetic hierarchy is studied, and found that even though the microorganisms are in the same genus, the expression of genes are different. In India, polyhydroxyalkanoate is synthesized using the phototrophic bacteria by Prof Paul, and In Canada, Prof Marchessault made a test on the biodegradability of polyhydroxyalkanoate material.

Polyhydroxyalkanoates have been developed and produced since the 1970s. Polyhydroxyalkanoate were produced using the natural soil microorganisms through the fermentation process by the ICI company. At the same time, polyhydroxyalkanoates were produced using the microorganism and by the engineering technique at Massachusetts Institute of Technology (MIT), and this results in the formation of Metabolix company in 1992. The patent of the method for producing polyhydroxyalkanoate of the ICI company was assigned to Zeneca company, and was further assigned to the Monsanto company. Metabolix company purchased the related patents of polyhydroxyalkanoate from Monsanto company in 2001, and further developed them. The Metabolix company signed a contract with the Archer Daniels Midland (ADM) company in 2004 for large-scale producing biodegradable plastic polyhydroxyalkanoate with the 50000 tons of production capacity from the end of the 2007 to the beginning of the 2008. The production cost of using the microorganism fermentation method is about USD3-5/kg, but the cost for large scale production of biodegradable plastic polyhydroxyalkanoate can be reduced to USD 2-3/kg. The objective of the Metabolix company is to find a new way to produce polyhydroxyalkanoate at a production cost lower than USD 1-2/kg.

Taxonomy of Ralstonia eutropha strain includes Bacteria, Proteobacteria, Betaproteobacteria, Burkholderiales, Burkholderiaceae, and Cupravidus. Some are the same strains but have other names, such as Cupriavidus necator, Wautersia eutropha, and Ralstonia eutropha including Ralstonia eutropha JMP 134, Ralstonia eutropha H850, and Ralstonia eutropha H16. They are capable of metabolizing glycerol in order to synthesize polyhydroxyalkanoate.

The cost of carbon source and antibiotic in the fermentation medium for the synthesis of polyhydroxyalkanoate is the highest one. The cost of the carbon source and the nitrogen source is up to 80% based on the cost of the total materials. The cost of the carbon source and the nitrogen source is up to 50% to 80% based on the operation cost. The cost of the carbon source and the nitrogen source is so high, and thereby that becomes a major barrier for the development and commercialization of polyhydroxyalkanoate. Furthermore, the yield of polyhydroxyalkanoate is not high, and thereby there is a need to provide a method for synthesizing polyhydroxyalkanoate to increase the yield of polyhydroxyalkanoate and reduce the production cost.

### SUMMARY OF THE INVENTION

An objective of the present invention is to provide a method for synthesizing polyhydroxyalkanoate using a microorganism, and the method of the present invention comprises a step of providing a microorganism, a step of providing a fermentation tank, a step of fermentation and culture, and a step of taking out.

In the step of providing the microorganism, Ralstonia eutropha strain is provided. In the step of providing the fermentation tank, the fermentation tank is provided, into which the crude glycerol is added, and also (NH₄)₂SO₄, urea, NH₄Cl and NaNO₃ are added in order to adjust a C/N (carbon-to-nitrogen) ratio from 20:1 to 150:1 in the fermentation tank. Then, a pH value in the fermentation tank is adjusted to 5-9. The temperature of the fermentation tank is controlled at 20 to 45° C.

In the step of fermentation and culture, 5 to 20% by volume of Ralstonia eutropha strain is added into the fermentation tank, and a mixing speed of from 100 to 500 rpm, an aeration quantity of 0.5-2 vvm and a concentration of dissolved oxygen of 30 to 80% are used, and the fermentation process is carried out for 48 to 96 hours to allow Ralstonia eutropha strain to proliferate, and meanwhile to allow polyhydroxyalkanoate to be synthesized from the crude glycerol in Ralstonia eutropha strain. In the step of taking out, the Ralstonia eutropha strain in the fermentation tank is taken out, and polyhydroxyalkanoate is then produced by disrupting the cells of Ralstonia eutropha strain, followed by extracting polyhydroxyalkanoate out from the disrupted cells of Ralstonia eutropha strain.

In the method of the present invention, the crude glycerol byproduct of biodiesel production or an industrial grade glycerol is used as a carbon source, and thereby the production cost can be reduced. The microorganisms are forced to metabolize under specific conditions resulting in an increase in the amount of the polyhydroxyalkanoate synthesized from glycerol in the microorganisms. In addition, the starting material and the synthesized product are all environmentally friendly, and thereby these materials are good for the sustainable development of natural resources.

### BRIEF DESCRIPTION OF THE DRAWINGS

The present invention will be apparent to those skilled in the art by reading the following detailed description of a preferred embodiment thereof, with reference to the attached drawing, in which:

FIG. 1 is a flowchart illustrating a method for synthesizing polyhydroxyalkanoate using a microorganism according to the present invention.

### DETAILED DESCRIPTION OF THE PREFERRED EMBODIMENT

The accompanying drawing is included to provide a further understanding of the invention, and is incorporated in and constituted a part of this specification. The drawing illustrates one embodiment of the invention and, together with the description, serves to explain the principles of the invention.

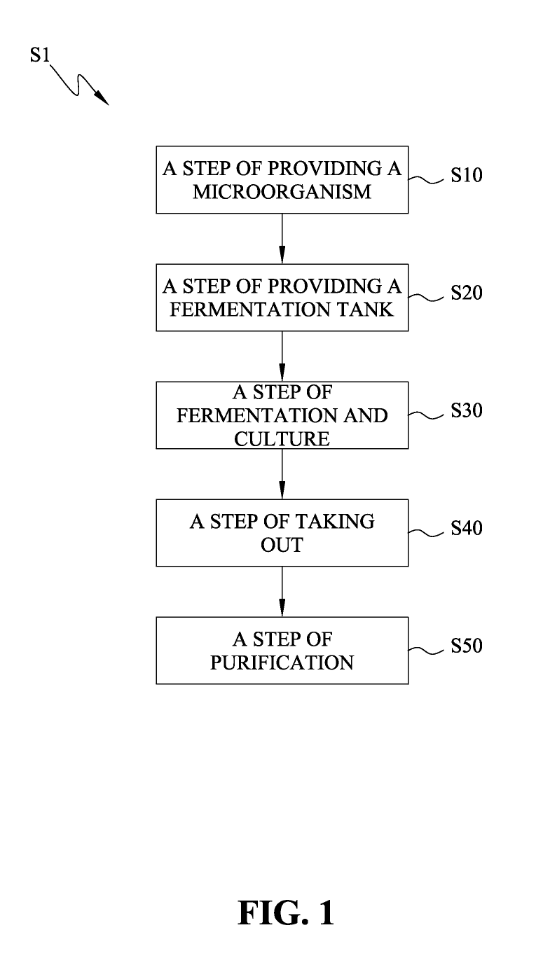

FIG. 1 is a flowchart illustrating a method for synthesizing polyhydroxyalkanoate using a microorganism according to the present invention. Referring to FIG. 1, a method for synthesizing polyhydroxyalkanoate using a microorganism S1 according to the present invention includes a step of providing a microorganism S10, a step of preparing a fermentation tank S20, a step of fermentation and culture S30, and a step of taking out S40.

In the step of providing the microorganism S10, Ralstonia eutropha strain is provided. In the step of providing the fermentation tank S20, the fermentation tank is provided, into which the crude glycerol is added, and also (NH₄)₂SO₄, urea, NH4Cl and NaNO3 are added in order to adjust a C/N (carbon-to-nitrogen) ratio in the fermentation tank, wherein the preferred C/N ratio is 20:1 to 150:1. Then, a pH value in the fermentation tank is adjusted by adding 2N H₂SO₄ and 2N NaOH into the fermentation tank, and the preferred pH value is 5-9. The temperature of the fermentation tank is controlled at 20 to 45° C.

Ralstonia eutropha strain is commercially available, and is purchased from Food Industry Research and Development Institute in Taiwan (ATCC catalog number: 17699, BCRC number: 13036).

In the step of fermentation and culture S30, 5 to 20% by volume of Ralstonia eutropha strain is added into the fermentation tank, and a mixing speed of from 100 to 500 rpm, an aeration quantity of 0.5-2 vvm and a concentration of dissolved oxygen of 30 to 80% are used, and the fermentation process is carried out for 48 to 96 hours to allow Ralstonia eutropha strain to proliferate, and meanwhile to allow polyhydroxyalkanoate to be synthesized from the crude glycerol in Ralstonia eutropha strain. In the step of taking out S40, the Ralstonia eutropha strain in the fermentation tank is taken out, and polyhydroxyalkanoate is then produced by disrupting the cells of Ralstonia eutropha strain, followed by extracting polyhydroxyalkanoate out from the disrupted cells of Ralstonia eutropha strain.

Furthermore, the method for synthesizing polyhydroxyalkanoate using a microorganism according to the present invention can further include a step of purifying S50 in which polyhydroxyalkanoate obtained from the step of taking out S40 can be purified.

In the method of the present invention, the crude glycerol byproduct of biodiesel production or an industrial grade glycerol is used as a carbon source, and thereby the production cost is reduced. The microorganisms are forced to metabolize under specific conditions resulting in an increase in the amount of the polyhydroxyalkanoate synthesized from glycerol in the microorganisms. In addition, the starting material and the synthesized product are all environmentally friendly, and thereby these materials are good for the sustainable development of natural resources.

Although the present invention has been described with reference to the preferred embodiments thereof, it is apparent to those skilled in the art that a variety of modifications and changes may be made without departing from the scope of the present invention which is intended to be defined by the appended claims.

1. A method for synthesizing polyhydroxyalkanoate using a microorganism, comprising:
providing a Ralstonia eutropha strain;
providing a fermentation tank and adding crude glycerol into the fermentation tank;
adding a nitrogen source into the fermentation tank to adjust a carbon to nitrogen C/N ratio to 20:1 to 150:1
controlling temperature of the fermentation tank to a temperature ranging from 20 to 45° C.;
adding 5 to 20% by volume of the Ralstonia eutropha strain into the fermentation Tank using a mixing speed of from 100 to 500 rpm, an aeration quantity of 0.5-2 vvm and a concentration of dissolved oxygen of 30 to 80%;
fermenting contents of the fermentation tank for 48 to 96 hours to allow the Ralstonia eutropha strain to proliferate and to allow polyhydroxyalkanoate to be synthesized from the crude glycerol within cells of the Ralstonia eutropha strain; and
disrupting the cells of the Ralstonia eutropha strain and extracting polyhydroxyalkanoate there from.

2. The method of claim 1, wherein the nitrogen source is at least one of (NH₄)₂SO₄, urea, NH₄Cl and NaNO₃.

3. The method of claim 1, further comprising adding 2N H₂SO₄ and 2N NaOH into the fermentation tank to adjust pH of the contents to a value of 5-9.

4. The method of claim 1, further comprising purifying the extracted polyhydroxyalkanoate.

5. The method of claim 1, wherein the crude glycerol is a crude glycerol byproduct of biodiesel production or an industrial grade glycerol and is used as a carbon source.
